(19) United States
(12) Patent Application Publication
(10) Pub. No.: US 2003/0165547 A1
(43) Pub. Date: Sep. 4, 2003

(54) Cosmetic uses of 3beta-acetoxy-7-oxo-DHEA

(76) Inventors: Elisabeth Picard-Lesboueyries, Velizy (FR); Beatrice Renault, Saint Maurice (FR); Veronique Burnier, Paris (FR); Roland Roguet, Paris (FR); Guy Courchay, Sucy-en-Brie (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/147,355

(22) Filed: May 17, 2002

Related U.S. Application Data
(60) Provisional application No. 60/307,837, filed on Jul. 27, 2001.

(30) Foreign Application Priority Data
May 18, 2001 (FR) 01 06604

(51) Int. Cl. A61K 7/00

(52) U.S. Cl. 424/401

(57) ABSTRACT

The present invention relates to the cosmetic use of 3β-acetoxy-7-oxo-DHEA to improve the appearance of keratin materials, with the exception of the treatment of wrinkles and fine lines, dry skin and dermatitis. This use is intended in particular for preventing or treating the loss of firmness of the skin and/or a dull complexion and/or dilation of the pores and/or skin or hair pigmentation disorders and/or hyperseborrhea and/or hyperseborrhea-related imperfections and/or sensitive skin and/or dandruff and/or hair loss and/or baldness.

The invention also relates to a cosmetic composition containing, in a physiologically acceptable medium, 3β-acetoxy-7-oxo-DHEA in combination with at least one other compound.

The present invention relates to the cosmetic use of 3β-acetoxy-7-oxo-DHEA to improve the appearance of keratin materials, with the exception of the treatment of wrinkles and fine lines, dry skin and dermatitis. This use is intended in particular for preventing or treating the loss of firmness of the skin and/or a dull complexion and/or dilation of the pores and/or skin or hair pigmentation disorders and/or hyperseborrhea and/or hyperseborrhea-related imperfections and/or sensitive skin and/or hair loss and/or baldness. The invention also relates to a cosmetic composition containing, in a physiologically acceptable medium, 3β-acetoxy-7-oxo-DHEA in combination with at least one other compound.

DHEA, or dehydroepiandrosterone, is a natural steroid produced essentially by the adrenal glands. Exogenous DHEA, administered topically or orally, is known for its capacity to promote epidermal keratinization (JP-07 196 467) and to treat dry skin by increasing the endogenous production and secretion of sebum and thus reinforcing the skin's barrier effect (U.S. Pat. No. 4,496,556). Patent U.S. Pat. No. 5,843,932 has also described the use of DHEA to remedy dermal atrophy by inhibiting the loss of collagen and connective tissue. Finally, the Applicant has demonstrated the capacity of DHEA to combat the weathered appearance of the skin (FR 00/00349), to modify skin and hair pigmentation (FR 99/12773) and to combat epidermal atrophy (FR 00/06154). These properties of DHEA make it a candidate of choice as an anti-ageing active agent.

However, for regulatory reasons, the hormonal nature of DHEA currently prevents it from being used for the manufacture of cosmetic products in certain countries, and there is thus still a need to provide DHEA analogues with properties as advantageous as DHEA itself, but without any hormonal effects.

Now, it appeared to the Applicant that 3-acetoxy-7-oxo-DHEA, or Δ5-androstene-3β-acetoxy-7,17-dione, could satisfy this need.

This compound has already been described as being effective in modulating the immune system (U.S. Pat. No. 5,292,730; U.S. Pat. No. 5,585,371; U.S. Pat. No. 5,641,766), treating Alzheimer's disease (U.S. Pat. No. 5,707,983) and treating HIV syndrome (U.S. Pat. No. 5,885,977) and for promoting weight loss (U.S. Pat. No. 5,296,481; U.S. Pat. No. 5,807,848).

Document WO 99/25333 also mentions the use, especially the topical use, of 3β-acetoxy-7-oxo-DHEA in the prophylactic and curative treatment of lupus erythematosus, which is a disorder of the immune system that is liable to affect several organs and that is manifested in the skin by transverse redness of the face and/or by squamous erythemal plaques disseminated over the body.

However, to the Applicant's knowledge, it has never been proposed to use 3β-acetoxy-7-oxo-DHEA for cosmetic purposes.

The present invention thus relates to the cosmetic use of 3β-acetoxy-7-oxo-DHEA to improve the appearance of keratin materials, with the exception of the treatment of wrinkles and fine lines, dry skin and dermatitis, in particular for preventing or treating the loss of firmness of the skin and/or a dull complexion and/or dilation of the pores and/or skin or hair pigmentation disorders and/or hyperseborrhea and/or hyperseborrhea-related imperfections and/or sensitive skin and/or dandruff and/or hair loss and/or baldness.

3β-Acetoxy-7-oxo-DHEA may be prepared, for example, according to the process described in patents U.S. Pat. No. 5,869,709 and U.S. Pat. No. 6,111,118.

The present invention also relates to a cosmetic composition containing, in a physiologically acceptable medium, 3β-acetoxy-7-oxo-DHEA and at least one compound chosen from: a desquamating agent, a moisturizer, a depigmenting or pro-pigmenting agent, an anti-glycation agent, an NO-synthase inhibitor, a 5α-reductase inhibitor, a lysyl and/or prolyl hydroxylase inhibitor, an agent for stimulating the synthesis of dermal or epidermal macromolecules and/or for preventing their degradation, an agent for stimulating the proliferation of fibroblasts or keratinocytes and/or keratinocyte differentiation, a muscle relaxant, an antimicrobial agent, a tensioning agent and an antipollution agent or free-radical scavenger.

The present invention also relates to a cosmetic composition containing, in a physiologically acceptable medium, 3β-acetoxy-7-oxo-DHEA and at least one given UV screening agent, chosen from certain UVA and/or UVB screening agents and/or at least one optionally coated mineral pigment.

Although it can be administered orally, the composition according to the invention is preferably suitable for topical application to keratin materials such as the skin, keratin fibers (hair and eyelashes) and the nails.

In any case, the composition according to the invention comprises an effective amount of DHEA derivative, sufficient to obtain the desired effect, and a physiologically acceptable medium.

To give an order of magnitude, the composition according to the invention can contain from 0.00001% to 10% by weight of 3β-acetoxy-7-oxo-DHEA relative to the total weight of the composition. Preferably, however, this composition will contain from 0.001% to 5% by weight of 3β-acetoxy-7-oxo-DHEA relative to the total weight of the composition.

The above composition may be used for cosmetic purposes, to improve the appearance of keratin materials.

This composition may thus be used to prevent or treat wrinkles and fine lines and/or the loss of firmness, tonicity and/or elasticity of the skin and/or a dull complexion and/or dilation of the pores and/or skin or hair pigmentation disorders and/or skin dryness and/or hyperseborrhea and/or hyperseborrhea-related imperfections and/or sensitive skin and/or dandruff and/or hair loss and/or baldness.

Various compounds that may be introduced into the composition according to the invention will now be detailed.

1. Desquamating Agents and Moisturizers

The term “desquamating agent” means any compound capable of acting:

either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;

or on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; amino-sulfonic compounds and in particular (N-2-hydroxyethylpiperazine-N-2-ethane)sulfonic acid (HEPES); 2-oxothiazolidine-4-carboxylic acid (procysteine) derivatives; α-amino acid derivatives of the type such as glycine (as described in EP-0 852 949); honey; sugar derivatives such as O-octanoyl-6-D-maltose and N-acetylglucosamine.

The term “moisturizer” means:

either a compound acting on the barrier function, in order to keep the stratum corneum moisturized, or an occlusive compound. Mention may be made of ceramides, sphingoid-based compounds, lecithins, glycosphingolipids, phospholipids, cholesterol and its derivatives, phytosterols (stigmasterol, β-sitosterol or campesterol), essential fatty acids, 1,2-diacyl-glycerol, 4-chromanone, pentacyclic triterpenes such as ursolic acid, petroleum jelly and lanolin;

or a compound that directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroyl-pyrrolidonecarboxylic acid and N-α-benzoyl-L-arginine;

or a compound that activates the sebaceous glands, such as steroid derivatives (including DHEA) and vitamin D and its derivatives.

These compounds can represent from 0.001% to 30% and preferably from 0.01% to 20% of the total weight of the composition according to the invention.

The composition according to the present invention comprising the desquamating agents and moisturizers mentioned above is advantageously intended for preventing or treating skin dryness and especially xerosis.

2. Depigmenting or Propigmenting Agent

The depigmenting agents that may be incorporated into the composition according to the present invention comprise, for example, the following compounds: kojic acid; ellagic acid; arbutin and its derivatives such as those described in patent applications EP-895 779 and EP-524 109; hydroquinone; aminophenol derivatives such as those described in patent applications WO 99/10318 and WO 99/32077, and in particular N-cholesteryloxycarbonyl-para-aminophenol and N-ethyloxycarbonyl-para-aminophenol; iminophenol derivatives, in particular those described in patent application WO 99/22707; L-2-oxothiazolidine-4-carboxylic acid or procysteine, and also its salts and esters; ascorbic acid and its derivatives, especially ascorbyl glucoside; and plant extracts, in particular extracts of liquorice, of mulberry and of skullcap, without this list being limiting.

Propigmenting agents that may be mentioned include the extract of burnet (Sanguisorba officinalis) sold by the company Maruzen, and extracts of chrysanthemum (Chrysanthemum morifolium).

The composition according to the present invention comprising the depigmenting agents mentioned above is advantageously intended for preventing or treating hyperpigmentation, in particular pigmentary marks related to ageing of the skin.

For its part, the composition containing the propigmenting agents mentioned above is preferably intended for treating baldness.

3. Anti-glycation Agent

The term “anti-glycation agent” means a compound for preventing and/or reducing the glycation of skin proteins, in particular of dermal proteins such as collagen.

Examples of anti-glycation agents are plant extracts of the Ericacea family, such as an extract of blueberry (Vaccinium angustifolium); ergothioneine and its derivatives; and hydroxystilbenes and their derivatives, such as resveratrol and 3,3′,5,5′-tetrahydroxystilbene. These anti-glycation agents are described in patent applications FR 99/16166, FR 00/08158, FR 99/09267 and FR 99/16168, respectively. Resveratrol is particularly preferred for use in this invention.

The composition according to the invention comprising an anti-glycation agent as defined above may be used advantageously to prevent or treat the signs of ageing of the skin, in particular to prevent or treat the loss of tonicity and/or elasticity of the skin.

4. NO-synthase Inhibitor

Examples of NO-synthase inhibitors that are suitable for use in the present invention especially comprise a plant extract of the species Vitis vinifera which is sold especially by the company Euromed under the name “Leucocyanidines de raisins extra”, or by the company Indena under the name Leucoselect®, or finally by the company Hansen under the name “Extrait de marc de raisin”; a plant extract of the species Olea europaea which is preferably obtained from olive tree leaves and is sold especially by the company Vinyals in the form of a dry extract, or by the company Biologia & Technologia under the trade name Eurol BT; and a plant extract of the species Gingko biloba which is preferably a dry aqueous extract of this plant sold by the company Beaufour under the trade name “Gingko biloba extrait standard”.

The composition according to the invention comprising an NO-synthase inhibitor as defined above may advantageously be used to prevent or treat the signs of ageing of the skin and/or sensitive skin.

5. 5α-reductase Inhibitor

When the composition according to the invention comprises a 5α-reductase inhibitor, this inhibitor may be chosen especially from:

retinoids, and in particular retinol;

sulfur and sulfur derivatives;

zinc salts such as zinc lactate, gluconate, pidolate, carboxylate, salicylate and/or cysteate;

selenium chloride;

vitamin B6 or pyridoxine;

mixture of capryloyl glycine, sarcosine and extract of Cinnamomum zeylanicum sold especially by the company SEPPIC under the trade name Sepicontrol A5®;

an extract of Laminaria saccharina sold especially by the company SECMA under the trade name Phlorogine®;

an extract of Spiraea ulmaria sold especially by the company Silab under the trade name Sebonormine®;

plant extracts from the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all sold, for example, by the company Maruzen;

an extract of Serenoa repens sold especially by the company Euromed;

plant extracts of the genus Silybum;

plant extracts containing sapogenins and in particular extracts of diosgenin-rich or hecogenin-rich Dioscorea plants; and

extracts of Eugenia caryophyllata containing eugenol or eugenyl glucoside.

The 5α-reductase inhibitor represents, for example, from 0.001% to 10% and preferably from 0.01% to 5% of the total weight of the composition according to the invention. When this composition contains such a compound, it is particularly suitable for preventing or treating seborrhea and/or hirsutism and/or androgen-dependent alopecia.

6. Lysyl and/or Prolyl Hydroxylase Inhibitor

Preferred examples of lysyl and/or propyl hydroxylase inhibitors that may be used in the composition according to the present invention are 2,4-diaminopyrimidine 3-oxide or 2,4-DPO described in patent application WO 96/09048 and 2,4-diamino-6-piperidinopyrimidine 3-oxide or “Minoxidil” described in patents U.S. Pat. No. 4,139,619 and U.S. Pat. No. 4,596,812.

These compounds are present, for example, in the composition according to the invention in a proportion of from 0.001% to 5% by weight and better still in a proportion of from 0.01% to 5% by weight relative to the total weight of the composition.

The composition containing the lysyl and/or prolyl hydroxylase inhibitor and the DHEA derivative according to the invention is advantageously used for preventing or treating alopecia.

7. Agent for Stimulating the Synthesis of Dermal or Epidermal Macromolecules and/or for Preventing their Degradation

Among the active agents for stimulating dermal macromolecules, mention may be made of those that act:

either on collagen synthesis, such as extracts of Centella asiatica; asiaticosides and derivatives; ascorbic acid or vitamin C and its derivatives; synthetic peptides such as iamin, biopeptide CL or the palmitoyloligopeptide sold by the company Sederma; peptides extracted from plants, such as the soybean hydrolyzate sold by the company Coletica under the trade name Phytokine®; and plant hormones such as auxins;

or on elastin synthesis, such as the extract of Saccharomyces cerivisiae sold by the company LSN under the trade name Cytovitin®; and the extract of the alga Macrocystis pyrifera sold by the company SECMA under the trade name Kelpadelie®;

or on glycosaminoglycan synthesis, such as the product of fermentation of milk with Lactobacillus vulgaris, sold by the company Brooks under the trade name Biomin yogourth®; the extract of the brown alga Padina pavonica sold by the company Alban Müller under the trade name HSP3®; and the extract of Saccharomyces cerevisiae available especially from the company Silab under the trade name Firmalift® or from the company LSN under the trade name Cytovitin®;

or on fibronectin synthesis, such as the extract of the zooplankton Salina sold by the company Seporga under the trade name GP4G®;

the yeast extract available especially from the company Alban Müller under the trade name Drieline®; and the palmitoyl pentapeptide sold by the company Sederma under the trade name Matrixil®;

or on the inhibition of metalloproteases (MMP), such as, more particularly, MMP 1, 2, 3 or 9. Mention may be made of: retinoids and derivatives, isoflavonoids, oligopeptides and lipopeptides, lipoamino acids, the malt extract sold by the company Coletica under the trade name Collalift®; extracts of blueberry or of rosemary; lycopene; isoflavones, their derivatives or plant extracts containing them, in particular extracts of soybean (sold, for example, by the company Ichimaru Pharcos under the trade name Flavosterone SB®), of red clover, of flax, of kakkon, or of sage;

or on the inhibition of serine proteases such as leukocyte elastase or cathepsin G. Mention may be made of: the peptide extract of Leguminosa seeds (Pisum sativum) sold by the company LSN under the trade name Parelastyl®; and heparinoids and pseudodipeptides.

Among the active agents that stimulate epidermal macromolecules, such as fillagrin and keratins, mention may be made especially of the extract of lupin sold by the company Silab under the trade name Structurine®; the extract of beech Fagus sylvatica buds sold by the company Gattefosse under the trade name Gatuline®; and the extract of the zooplankton Salina sold by the company Seporga under the trade name GP4G®.

The composition according to the invention containing one or more of the above compounds is particularly suitable for use in preventing or treating the signs of ageing of the skin, in particular loss of firmness and/or elasticity of the skin.

8. Agent for Stimulating the Proliferation of Fibroblasts or Keratinocytes and/or Keratinocyte Differentiation

The agents for stimulating the proliferation of fibroblasts that may be used in the composition according to the invention may be chosen, for example, from plant proteins or polypeptides, extracted especially from soybean (for example an extract of soybean sold by the company LSN under the name Eleseryl SH-VEG 8® or sold by the company Silab under the trade name Raffermine®); and plant hormones such as giberrellins and cytokinins.

The agents for stimulating keratinocyte proliferation that may be used in the composition according to the-invention especially comprise retinoids such as retinol and its esters, including retinyl palmitate; extracts of walnut cakes sold by the company Gattefosse; and extracts of Solanum tuberosum sold by the company Sederma.

The agents for stimulating keratinocyte differentiation comprise, for example, minerals such as calcium; the extract of lupin sold by the company Silab under the trade name Photopreventine®; sodium β-sitosteryl sulfate sold by the company Seporga under the trade name Phytocohesine®; and the extract of corn sold by the company Solabia under the trade name Phytovityl®.

The composition according to the invention comprising these compounds is preferably intended to be used for preventing or treating the signs of ageing of the skin.

9. Muscle Relaxant

The muscle relaxants that may be used in the composition according to the invention comprise calcium inhibitors such as alverine and its salts, chlorine-channel openers such as Diazepam, and catecholamine and acetylcholine inhibitors, such as the hexapeptide argireline R sold by the company Ilipotec.

The composition according to the invention comprising these compounds is preferably intended to be used for preventing or treating the signs of ageing of the skin and in particular wrinkles.

10. Antimicrobial Agent

The antimicrobial agents that may be used in the composition according to the invention may be chosen especially from 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in patent WO 93/18743, farnesol and phytosphingosines, and mixtures thereof.

The preferred antimicrobial agents are triclosan, phenoxyethanol, octoxyglycerine, octanoylglycine, 10-hydroxy-2-decanoic acid, caprylyl glycol, farnesol and azelaic acid.

By way of example, the antimicrobial agent may be used in the composition according to the invention in an amount representing from 0.1% to 20% and preferably from 0.1% to 10% of the total weight of the composition.

The composition containing 3-acetoxy-7-oxo-DHEA and the antimicrobial agent is particularly suitable for use in treating acne-prone greasy skin, acne or dandruff of the scalp.

11. Tensioning Agent

The term “tensioning agent” means a compound capable of exerting tension on the skin, the effect of which is to temporarily fade out irregularities on the skin's surface, such as wrinkles and fine lines.

Among the tensioning agents that may be used in the composition according to the present invention, mention may be made especially of:

(1) polyurethane latices or acrylic-silicone latices, in particular those described in patent application EP-1 038 519, such as a propylthio(polymethyl acrylate), propylthio(polymethyl methacrylate) and propylthio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are sold especially by the company 3M under the trade names VS 80, VS 70 or LO21.

(2) soybean or wheat plant proteins, and/or

(3) sodium magnesium silicates (Laponites).

The compositions according to the invention comprising the above tensioning agents are advantageously intended for treating the signs of ageing of the skin, in particular wrinkles and fine lines.

12. Anti-pollution Agent or Free-radical Scavenger

The term “anti-pollution agent” means any compound capable of trapping ozone, monocyclic or polycyclic aromatic compounds such as benzopyrene and/or heavy metals such as cobalt, mercury, cadmium and/or nickel. The term “free-radical scavenger” means any compound capable of trapping free radicals.

As ozone-trapping agents that may be used in the composition according to the invention, mention may be made in particular of vitamin C and its derivatives including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chorogenic acid; stilbenes, in particular resveratrol; sulfur-containing amino acid derivatives, in particular S-carboxymethylcysteine; ergothioneine; N-acetylcysteine; chelating agents, for instance N,N′-bis(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenylalanine, tyrosine and hydrolyzed RNA, sold by the Laboratoires Sérobiologiques under the trade name CPP LS 2633-12F®, the water-soluble fraction of corn sold by the company Solabia under the trade name Phytovityl®, the mixture of extract of fumitory and of extract of lemon sold under the name Unicotrozon C-49® by the company Induchem, and the mixture of extracts of ginseng, of apple, of peach, of wheat and of barley, sold by the company Provital under the trade name Pronalen Bioprotect®.

As agents for trapping out monocyclic or polycyclic aromatic compounds, which may be used in the composition according to the invention, mention may be made in particular of tannins such as ellagic acid; indole derivatives, in particular 3-indolecarbinol; extracts of tea, in particular of green tea, extracts of water hyacinth or Eichhornia crassipes; and the water-soluble fraction of corn sold by the company Solabia under the trade name Phytovityl®.

Finally, as heavy-metal-trapping agents that may be used in the composition according to the invention, mention may be made in particular of chelating agents such as EDTA, the pentasodium salt of ethylenediaminetetramethylenephosphonic acid, and N,N′-bis(3,4,5-trimethoxybenzyl)ethylenediamine or one of the salts, metal complexes or esters thereof; phytic acid; chitosan derivatives; extracts of tea, in particular of green tea; tannins such as ellagic acid; sulfur-containing amino acids such as cysteine; extracts of water hyacinth (Eichhornia crassipes); and the water-soluble fraction of corn sold by the company Solabia under the trade name Phytovityl®.

The free-radical scavengers that may be used in the composition according to the invention comprise, besides certain anti-pollution agents mentioned above, vitamin E and its derivatives such as tocopheryl acetate; bioflavonoids; coenzyme Q10 or ubiquinone; certain enzymes, for instance catalase, superoxide dismutase, lactoperoxidase, glutathione peroxidase and quinone reductases; glutathione; benzylidenecamphor; benzylcyclanones; substituted naphthalenones; pidolates; phytanetriol; gamma-oryzanol; lignans; and melatonin.

The compositions according to the invention comprising the anti-pollution agents and/or free-radical scavengers above are advantageously intended for preventing or treating the signs of ageing of the skin, in particular wrinkles, and loss of firmness and elasticity of the skin. As a variant, they are intended for preventing or treating a dull complexion.

13. UVA and/or UVB Screening Agent and Optionally Coated Mineral Pigments

The composition according to the invention may contain one or more UV screening agents capable of screening out UVA and/or UVB radiation.

As compounds capable of screening out UVA radiation, mention may be made especially of:

(1) benzophenone derivatives, for example:

2,4-dihydroxybenzophenone (benzophenone-1);

2,2′,4,4′-tetrahydroxybenzophenone (benzophenone-2);

2-hydroxy-4-methoxybenzophenone (benzophenone-3), available from the company BASF under the trade name Uvinul M40;

2-hydroxy-4-methoxybenzophenone-5-sulfonic acid (benzophenone-4) and also its sulfonate form (benzophenone-5), available from the company BASF under the trade name Uvinul MS40;

2,2′-dihydroxy-4,4′-dimethoxybenzophenone (benzophenone-6);

5-chloro-2-hydroxybenzophenone (benzophenone-7);

2,2′-dihydroxy-4-methoxybenzophenone (benzophenone-8);

the disodium salt of 2,2′-dihydroxy-4,4′-dimethoxybenzophenone-5,5′-disulfonic acid (benzophenone-9);

2-hydroxy-4-methoxy-4′-methylbenzophenone (benzophenone-10);

benzophenone-11;

2-hydroxy-4-(octyloxy)benzophenone (benzophenone-12);

benzophenones 3 and 5 being preferred;

(2) triazine derivatives, and in particular 2,4-bis{[4-(2-ethylhexyloxy)-2-hydroxy]phenyl}-6-(4-methoxyphenyl)-1,3,5-triazine available from the companay Ciba Geigy under the trade name Tinosorb S and 2,2′-methylenebis[6-(2H-benzotriazol-2-yl)-4-(1,1,3,3-tetramethylbutyl)phenol] available from the company Ciba Geigy under the trade name Tinosorb M;

(3) benzene-1,4-bis(3-methylidene-10-camphorsulfonic acid), optionally in partially or totally neutralized form, and

(4) mixtures thereof.

As compounds capable of screening out UVB radiation, mention may be made especially of:

(1) salicylic acid derivatives, in particular homomenthyl salicylate and octyl salicylate;

(2) cinnamic acid derivatives, in particular 2-ethylhexyl p-methoxycinnamate, available from the company Givaudan under the trade name Parsol MCX;

(3) liquid β,β′-diphenylacrylate derivatives, in particular 2-ethylhexyl α-cyano-α,β′-diphenylacrylate, or octocrylene, available from the company BASF under the trade name Uvinul N539;

(4) p-aminobenzoic acid derivatives;

(5) 4-methylbenzylidenecamphor available from the company Merck under the trade name Eusolex 6300;

(6) 2-phenylbenzimidazole-5-sulfonic acid sold under the trade name “Eusolex 232” by the company Merck;

(7) 1,3,5-triazine derivatives, in particular:

2,4,6-tris[p-(2′-ethylhexyl-1′-oxycarbonyl)anilino]-1,3,5-triazine, available from the company BASF under the trade name Uvinul T150, and

the compound corresponding to the formula (II) below:

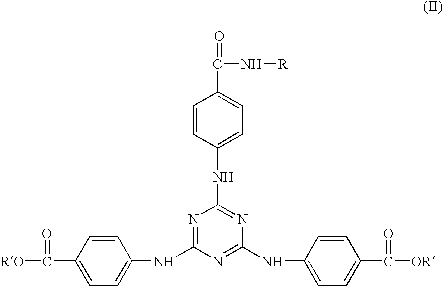

in which R′ denotes a 2-ethylhexyl radical and R denotes a tert-butyl radical, available from the company Sigma 3V under the trade name Uvasorb HEB;

(8) mixtures thereof.

As compounds capable of screening out UVA and UVB radiation, mention may be made in particular of the benzotriazole silicone corresponding to the general formula (III) below:

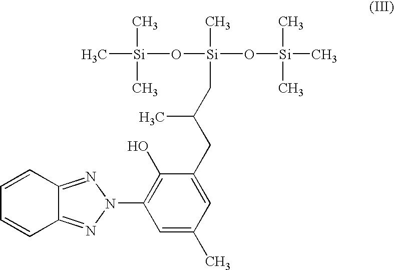

This benzotriazole silicone, and also the method for preparing it, are especially described in patent application FR-A-2 642 968.

As optionally coated mineral pigments, mention may be made of nanopigments of titanium oxide, of iron oxide, of zinc oxide, of zirconium oxide or of cerium oxide, optionally coated with alumina and/or with aluminum stearate.

In addition to the compound(s) described above, the composition according to the invention generally contains an effective amount of 3-acetoxy-7-oxo-DHEA, that is sufficient to obtain the desired effect. It thus contains, for example, from 0.00001% to 10% by weight of said 3-acetoxy-7-oxo-DHEA, relative to the total weight of the composition, and better still from 0.001% to 5% by weight of said 3-acetoxy-7-oxo-DHEA relative to the total weight of the composition.

The composition may be used for cosmetic purposes, to improve the appearance of keratin materials, in particular for preventing or treating wrinkles and fine lines and/or the loss of firmness, tonicity and/or elasticity of the skin and/or a dull complexion and/or dilation of the pores and/or skin or hair pigmentation disorders and/or skin dryness and/or hyperseborrhea and/or sensitive skin and/or dandruff and/or hair loss and/or baldness.

The composition according to the invention is preferably suitable for topical application to keratin materials such as the skin, the hair or the nails. It may be in any presentation form normally used for this type of application, especially in the form of an aqueous or oily solution, an oil-in-water or water-in-oil emulsion, a silicone emulsion, a microemulsion or nanoemulsion, an aqueous or oily gel or a liquid, pasty or solid anhydrous product.

This composition may be more or less fluid and may have the appearance of a white or colored cream, an ointment, a milk, a lotion, a serum, a paste, a mousse or a gel. It may optionally be applied to the skin in the form of an aerosol, a patch or a powder. It may also be in solid form and, for example, in the form of a stick. It may be used as a care product and/or as a makeup product for the skin. As a variant, it may be in the form of a shampoo or conditioner.

In a known manner, the composition of the invention may also contain adjuvants that are common in cosmetics, such as hydrophilic or lipophilic gelling agents, preserving agents, antioxidants, solvents, fragrances, fillers, pigments, odor absorbers and dyestuffs. The amounts of these various adjuvants are those conventionally used in the field under consideration, and for example from 0.01% to 20% of the total weight of the composition. Depending on their nature, these adjuvants may be introduced into the fatty phase or into the aqueous phase. These adjuvants, and the concentrations thereof, must be such that they do not harm the advantageous properties of the DHEA derivative according to the invention.

When the composition according to the invention is an emulsion, the proportion of the fatty phase can range from 5% to 80% by weight and preferably from 5% to 50% by weight relative to the total weight of the composition. The fatty substances, emulsifiers and coemulsifiers used in the composition in emulsion form are chosen from those conventionally used in the field under consideration. The emulsifier and coemulsifier are preferably present in the composition in a proportion ranging from 0.3% to 30% by weight and preferably from 0.5% to 20% by weight relative to the total weight of the composition.

As fatty substances that may be used in the invention, use may be made of oils and especially mineral oils (liquid petroleum jelly), oils of plant origin (avocado oil, soybean oil), oils of animal origin (lanolin), synthetic oils (perhydrosqualene), silicone oils (cyclomethicone) and fluoro oils (perfluoropolyethers). Fatty alcohols such as cetyl alcohol, fatty acids, waxes and gums and in particular silicone gums may also be used as fatty substances.

As emulsifiers and coemulsifiers that may be used in the invention, mention may be made, for example, of fatty acid esters of polyethylene glycol, such as PEG-100 stearate, PEG-50 stearate and PEG-40 stearate; fatty acid esters of polyols, such as glyceryl stearate, sorbitan tristearate and oxyethylenated sorbitan stearates available under the trade names Tween® 20 or Tween® 60, for example; and mixtures thereof.

Hydrophilic gelling agents that may be mentioned in particular include carboxyvinyl polymers (carbomer), acrylic copolymers such as acrylate/alkylacrylate copolymers, polyacrylamides, polysaccharides, natural gums and clays, and lipophilic gelling agents that may be mentioned include modified clays, for instance bentones, metal salts of fatty acids and hydrophobic silica.

According to another embodiment, the composition according to the invention may be suitable for oral administration. In this case, it may be, for example, in the form of syrups, suspensions, solutions, emulsions, granules, gel capsules, soft capsules or tablets.

The daily doses of 3β-acetoxy-7-oxo-DHEA administered orally can be, for example, up to 200 mg/day. They may be administered in a single dosage intake or in several dosage intakes, the dose per intake possibly being, for example, between 25 and 50 mg.

Finally, the present invention relates to a cosmetic process for treating keratin materials, with the exception of the treatment of wrinkles and fine lines, dry skin and dermatitis, comprising the oral administration or the topical application to the skin, the hair, the eyelashes or the nails of a composition containing 3β-acetoxy-7-oxo-DHEA in a physiologically acceptable medium.

The invention relates more particularly to a cosmetic process for treating the loss of firmness of the skin and/or a dull complexion and/or dilation of the pores and/or skin or hair pigmentation disorders and/or hyperseborrhea and/or hyperseborrhea-related imperfections and/or sensitive skin and/or dandruff and/or hair loss and/or baldness, comprising the oral administration or the topical application to the skin or the hair of a composition containing 3β-acetoxy-7-oxo-DHEA in a physiologically acceptable medium.

The invention will now be illustrated by the nonlimiting examples that follow. In these examples, the amounts are given as weight percentages.

### EXAMPLES

### Example 1

### Moisturizing Cream

| | |
|---|---|
| Phase A | |
| Acrylate/C₁₀₋₃₀ acrylate copolymer | 0.5% |
| Water | 12% |
| Phase B | |
| Hydrogenated polyisobutene | 5% |
| 3β-acetoxy-7-oxo-DHEA | 0.5% |
| Cyclohexasiloxane | 6% |
| Phase C | |
| Triethanolamine | 1% |
| Glycerol | 6% |
| EDTA | 0.2% |
| Preserving agents | 0.5% |
| Glycine | 2% |
| Polyacrylamide and C₁₃₋₁₄ isoparaffin and | 1% |
| laureth-7 | |
| Water | qs 100% |

This composition may be prepared in the following manner. The polymer of phase A is dispersed in water at 40° C. The constituents of phase B are heated to 70° C. until completely dissolved, and the temperature is then reduced to 40° C. The constituents of phase C are mixed together at 50° C. Phase B is then introduced into phase A at 40° C. with stirring, and phase C is then added thereto.

The above composition rehydrates dry skin and makes it smooth.

### Example 2

### Moisturizing Cream

The composition below is prepared in a standard manner for those skilled in the art.

| Phase A | |
|---|---|
| Demineralized water | qs 100% |
| Preserving agents | 0.5% |
| Carbomer | 0.4% |
| Glycerol | 7% |
| Phase B1 | |
| Oxyethylenated (200 EO) sorbitan stearate | 0.9% |
| Phase B2 | |
| PEG-100 stearate and glyceryl stearate | 2.1% |
| Isononyl isononanoate | 10% |
| Petroleum jelly | 2% |
| Octyldodecanol | 10% |
| 3β-acetoxy-7-oxo-DHEA | 0.2% |
| Butylhydroxytoluene | 0.1% |
| UV screening agent | 1% |
| Ceramides | 0.5% |
| Phase C | |
| Water | 2% |
| Triethanolamine | 0.5% |
| Urea | 1% |

This cream may be used to care for dry skin.

### Example 3

### Anti-ageing Cream

| | |
|---|---|
| Phase A | |
| Acrylate/C₁₀₋₃₀ acrylate copolymer | 0.5% |
| Water | 12% |
| Phase B | |
| Hydrogenated polyisobutene | 5% |
| 3β-acetoxy-7-oxo-DHEA | 0.5% |
| Cyclohexasiloxane | 5% |
| Octyl methoxycinnamate | 1% |
| Phase C | |
| Triethanolamine | 1% |
| Glycerol | 6% |
| Preserving agents | 0.5% |
| Polyacrylamide and C₁₃₋₁₄ isoparaffin and | 1% |
| laureth-7 | |
| Water | qs 100% |

This composition may be prepared in the following manner. The polymer of phase A is dispersed in water at 40° C. The constituents of phase B are heated to 70° C. until completely dissolved, and the temperature is then reduced to 40° C. The constituents of phase C are mixed together at 50° C. Phase B is then introduced into phase A at 40° C. with stirring, and phase C is then added thereto.

This cream may be used in application once or twice a day for treating the signs of ageing of the skin, and in particular for fading out wrinkles and fine lines.

### Example 4

### Anti-ageing Cream

The composition below is prepared in a standard manner for those skilled in the art.

| Phase A | |
|---|---|
| Demineralized water | qs 100% |
| Preserving agents | 0.5% |
| Carbomer | 0.4% |
| Glycerol | 7% |
| Phase B1 | |
| Oxyethylenated (200 EO) sorbitan stearate | 0.9% |
| Phase B2 | |
| PEG-100 stearate and glyceryl stearate | 2.1% |
| Isononyl isononanoate | 10% |
| Octyldodecanol | 10% |
| 3β-acetoxy-7-oxo-DHEA | 0.2% |
| Butylhydroxytoluene | 0.1% |
| UV screening agent | 1% |
| Phase C | |
| Water | 2% |
| Triethanolamine | 0.5% |
| Extract of Centella asiatica | 1% |
| Palmitoyl pentapeptide (Matrixyl ® from Sederma) | 0.1% |

This cream is useful as a firming day cream.

### Example 5

### Gel for Cleansing Greasy Skin

The composition below is prepared in a standard manner for those skilled in the art:

| | |
|---|---|
| Lauryl phosphate | 6.5% |
| Decyl glucoside | 16.25% |
| Polyquaternium-7 | 5.7% |
| Oxyethylenated (150 EO) pentaerythrityl | 0.5% |
| tetrastearate | |
| Glycerol | 3.5% |
| Sorbitol | 3.5% |
| Potassium hydroxide | 1.7% |
| Hydroxypropylcellulose | 0.2% |
| Disodium EDTA | 0.05% |
| Sodium chloride | 0.1% |
| 3β-acetoxy-7-oxo-DHEA | 0.1% |
| Preserving agents | 0.3% |
| Water | qs 100% |

This gel makes it possible to control the secretions of sebum and to attenuate skin imperfections.

### Example 6

### Anti-blemish Patch

A patch comprising the composition below is prepared:

| prepared: | |
|---|---|
| Water | 40% |
| Alcohol | qs |
| Glycerol | 7% |
| 3β-acetoxy-7-oxo-DHEA | 0.5% |
| Polyvinyl alcohol | 5% |
| Kojic acid | 0.5% |

This patch may be applied to the hands and the neckline to fade out pigmentary marks, in particular age marks.

### Example 7

### Lotion for Preventing Hair Loss

The composition below is prepared in a standard manner for those skilled in the art:

| | |
|---|---|
| Water | 25% |
| Glycerol | 7% |
| 3β-acetoxy-7-oxo-DHEA | 0.5% |
| Alcohol | qs 100% |

This lotion is effective for preventing hair loss.

### Example 8

### Anti-ageing Food Supplement

Soft capsules having the composition below are prepared in a standard manner for those skilled in the art:

| | | |
|---|---|---|
| Hydrogenated soybean oil | 40 | mg |
| Wheat oil | 95 | mg |
| Soybean lecithin | 20 | mg |
| Natural tocopherols | 5 | mg |
| Ascorbic acid | 30 | mg |
| Soybean extract available under the name | 120 | mg |
| Novasoy ® from the company AMD | | |
| (corresponding to about 50 mg of isoflavones) | | |
| 3β-acetoxy-7-oxo-DHEA | 50 | mg |

1. The cosmetic use of 3β-acetoxy-7-oxo-DHEA to improve the appearance of keratin materials, with the exception of the treatment of wrinkles and fine lines, dry skin and dermatitis.

2. The cosmetic use of 3β-acetoxy-7-oxo-DHEA for preventing or treating the loss of firmness of the skin and/or a dull complexion and/or dilation of the pores and/or skin or hair pigmentation disorders and/or hyperseborrhea and/or hyperseborrhea-related imperfections and/or sensitive skin and/or dandruff and/or hair loss and/or baldness.

3. A cosmetic composition containing, in a physiologically acceptable medium, 3β-acetoxy-7-oxo-DHEA and at least one compound chosen from: a desquamating agent, a moisturizer, a depigmenting or propigmenting agent, an anti-glycation agent, an NO-synthase inhibitor, a 5α-reductase inhibitor, a lysyl and/or prolyl hydroxylase inhibitor, an agent for stimulating the synthesis of dermal or epidermal macromolecules and/or for preventing their degradation, an agent for stimulating keratinocyte proliferation and/or differentiation, a muscle relaxant, an antimicrobial agent, a tensioning agent, an anti-pollution agent or a free-radical scavenger.

4. A cosmetic composition containing, in a physiologically acceptable medium, 3β-acetoxy-7-oxo-DHEA and at least one optionally coated mineral pigment and/or at least one UV screening agent chosen from:
(a) a benzophenone derivative;
(b) a triazine derivative;
(c) benzene-1,4-bis(3-methylidene-10-camphorsulfonic acid), optionally in partially or totally neutralized form;
(e) a salicylic acid derivative;
(f) a cinnamic acid derivative;
(g) a liquid β,β′-diphenylacrylate derivative;
(h) a p-aminobenzoic acid derivative;
(i) 4-methylbenzylidenecamphor;
(j) 2-phenylbenzimidazole-5-sulfonic acid;
(k) a 1,3,5-triazine derivative;
(l) a benzotriazole silicone of formula:

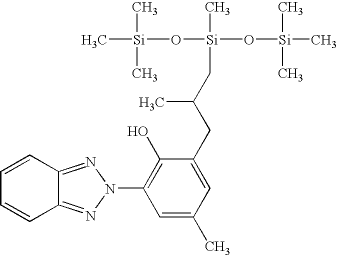

5. The composition as claimed in claim 3 or 4, characterized in that it is suitable for topical application to keratin materials.

6. The composition as claimed in claim 5, characterized in that the keratin materials are chosen from: the skin, the hair, the eyelashes and the nails.

7. The composition as claimed in any one of claims 3 to 6, characterized in that it contains from 0.00001% to 10% by weight of 3β-acetoxy-7-oxo-DHEA relative to the total weight of the composition.

8. The composition as claimed in claim 7, characterized in that it contains from 0.001% to 5% by weight of 3β-acetoxy-7-oxo-DHEA relative to the total weight of the composition.

9. The composition as claimed in claim 3, characterized in that it is suitable for oral administration.

10. The cosmetic use of the composition as claimed in any one of claims 3 to 9, for improving the appearance of keratin materials.

11. The cosmetic use of the composition as claimed in any one of claims 3 to 9, for preventing or treating wrinkles and fine lines and/or the loss of firmness, tonicity and/or elasticity of the skin and/or a dull complexion and/or dilation of the pores and/or skin or hair pigmentation disorders and/or skin dryness and/or hyperseborrhea and/or sensitive skin and/or dandruff and/or hair loss and/or baldness.

12. A cosmetic process for treating keratin materials, with the exception of the treatment of wrinkles and fine lines, dry skin and dermatitis, comprising the oral administration or the topical application to the skin, the hair, the eyelashes or the nails of a composition containing 3β-acetoxy-7-oxo-DHEA in a physiologically acceptable medium.

13. A cosmetic process for treating loss of firmness of the skin and/or a dull complexion and/or dilation of the pores and/or skin or hair pigmentation disorders and/or hyperseborrhea and/or sensitive skin and/or dandruff and/or hair loss and/or baldness, comprising the oral administration or topical application to the skin or the hair of a composition containing 3β-acetoxy-7-oxo-DHEA in a physiologically acceptable medium.
